(19) United States
(12) Patent Application Publication
(10) Pub. No.: US 2008/0294117 A1
(43) Pub. Date: Nov. 27, 2008

(54) Flexible tube coupler

(76) Inventor: DeAnna Ware, Seaside, CA (US)

Correspondence Address: Walt Froloff, 273D Searidge Rd, Aptos, CA, 95003, US

(21) Appl. No.: 11/805,755

(22) Filed: May 23, 2007

(51) Int. Cl. A61M 5/32

(52) U.S. Cl. 604/174

(57) ABSTRACT

The present invention is a tube coupler for securing flexible tubing in patient care, tubes used on patients for various purposes mostly medical, which are periodically removed, re-inserted or repositioned. A device formed from a base element with adjustable clamping elements and a mechanism for anchoring tube clamping elements for a friction fit around tubes is introduced.

### BACKGROUND

### Field of the Invention

The present invention generally relates to medical care flexible tubing for gas and fluid transport, and in particular to the stable holding of the tubing which are used for various purposes on patients, for external affixation and re-affixation due to periodic tube removal in patient care as they apply to the current medical needs and practices.

### Medical Care Flexible Tubing

Health care and medical patients in both emergent and non-emergent situations may require the use of flexible tubing for various reasons and purposes. Many circumstances require the use of more than on tube. An example of a multiple tube use is the simultaneous and complementary use of an Orogastric tube and Endotracheal tube.

Patients who require bag-and-mask ventilation for long periods of time often require insertion of an Orogastric tube for transferring gas and fluids to and from the stomach. Under some circumstances, air/gas pumped into the oropharynx also introduces free air to both the trachea and the esophagus. Some air enters the lungs and some is forced into the stomach. Gas forced into the stomach interferes with ventilation in a various of ways: 1) The stomach becomes distended thus putting pressure on the diaphragm and preventing the lungs from fully distending, 2) Gas in the stomach may also cause regurgitation of gastric contents, posing a risk of aspiration into the lungs during bag-and-mask ventilation.

A result of gastric/abdominal distension and aspiration of gastric contents can be reduced by inserting an Orogastric tube, suctioning gastric contents, and leaving the gastric tube in place to act as a vent for gas throughout the remainder of the resuscitation.

Whereas an Endotracheal tube is inserted to maintain a patent airway for ventilation purposes during resuscitation and also to administer medication. In neonates, Endotracheal tubes are used to clear meconium, for surfactant administration, and suspected diaphragmatic hernia. Therefore, often it is necessary to use an Orogastric tube in addition to a endotracheal tube concurrently and for different purposes, which are sometime causal.

Although there are flexible tubing anchors, the current practice is to attach the Endotracheal tube and the Orogastric tube together with tape. The bundle is then tape secured to patients to maintain stability. Generally, patients require various care, including chest x-rays, ordinary monitoring re-placement and repositioning, and or feeding and oral attention. Thus the Endotracheal tube, Nasogastric and Orogastric tube positions have a tendency to get displaced and thus requiring re-taping. Re-taping becomes problematic, as the tube relative positions may not be correctly re-positioned, or securely anchored to the patient in a comfortable manner. However, taping and re-taping is the normal practice. This is both wasteful of tape, gloves, tube resources and is also time consuming. In attempts to maintain the patients skin integrity, the Endotracheal tube must be re-taped and repositioned regularly. Applying and re-applying the taped Orogastric or Nasogastric tube to the Endotracheal tube causes the Endotracheal tube to become sticky, obscuring the numbers and meter lines painted on the tube for placement purposes. With sufficient re-tapes, the numbers and lines disappear. Caregivers gloves stick to the sticky tubes, causing care givers gloves to adhere to the sticky tubes during a re-taping, tears gloves and putting the caregiver at risk from exposure to the patients body fluids. This is also generally accompanied with cursing, obtaining new gloves, cleaning tubes, re-measuring, re-positioning and re-taping the tubes to patient.

Some cases of difficult airway intubation can be managed by using the Endotrol (trigger) Endotracheal tube. The management of a difficult airway can be facilitated initially by using the Endotrol tube; however, significant occlusion can occur later in time, when the tube “kinks,” leading to its partial collapse. The Endotrol tube has been used to maintain airways in critical and difficult situations. In other situations, kinking of the Lasertubus may result in occlusion of the Endotracheal tube, which in turn precluded adequate ventilation. Thus tubes can be removed entirely, replaced or be re-positioned during procedures.

Some related solutions for medical or health care are patient bracelets having catheters integrally formed therewith, securing the bracelet about the arm, wrist, on hand, etc. Others teach tube fitting anchoring to securely anchor a catheter and fluid supply tube interconnection to a patient's skin. Others claim internal tissue-anchoring devices inserted into a tissue or secured onto a tissue. Still others disclose a tube holder assembly for securing an endotracheal tube to a biteline of a patient's mouth.

Some claim methods for guiding an instrument associated with a medical device, such as an endoscope, for positioning a flexible medical instrument extending from an instrument channel of an endoscope, such as for the treatment of tissue. Still others disclose a workpiece engraving machine for a way to clamp, align and identify a workpiece for engraving.

None of these satisfactorily address the above problems with tube affixation and re-affixation, with multiple tubes for various purposes in standard practice.

What is needed are ways to couple medical tubes in or out patient care facilities wherein they can be secured stable yet removable and re-attachable. What are needed are quick tube securing and un-securing mechanisms, for use in emergent and non-emergent situations, for use with adults, pediatrics and neonates and without anymore training, to replace the current inefficient and wasteful practice of taping tubes together and to a patient. What are needed are devices for re-attaching tubes more precisely in their original positions after detachment, a device that reduces the need for taping tubes together. What is needed are ways that tubes can be disengaged and re-engaged to a patient with minimal chance for error of miss-positioning the inserted tube. What is needed are less wasteful methods of stabilizing tubes feeding or siphoning bodily fluids, oxygen, nutrients, etc. of patients. What are needed are ways to make care giving less guess work, error prone and easier, less costly and more precise.

### SUMMARY

The present invention discloses a tube coupler, for connecting and stably holding tubes used in patient care, where the tubes can be obtrusive and an impediment to care. Where tubes are re-positioned or removed and re-inserted and the transition causes potential problems in correct tube re-positioning or re-insertion depth. A flexible tube coupler comprising a base element, clamping elements coupled to base, clamping elements formed with curvature to partially surround a tubular object in a frictional fit, and a clamp element base position securing mechanism whereby at least one flexible tube can be wedged between the clamping elements and rigidly coupled to the base element, which is secured relative to the patient. The tube coupler may have slidably adjustable tube contour clamping elements on a base element slider for adjusting to and holding firmly different outside diameter flexible tubes, and may be made of various low cost materials.

### BRIEF DESCRIPTION OF DRAWINGS

FIG. 1 is a isometric view of a tube coupler in accordance with an embodiment of the present invention.

FIG. 2 is a side view drawing of a tube coupler in accordance with an embodiment of the present invention.

FIG. 3 is an isometric drawing of a tube coupler on belt in accordance with an embodiment of the present invention.

FIG. 4 is a side view drawing of a tube coupler secured to a belt in accordance with an embodiment of the present invention.

FIG. 5 is an isometric illustration of a one-piece clamping element coupler in accordance with an embodiment of the present invention.

FIG. 6 is an isometric illustration of a clasping tube coupler in accordance with an embodiment of the present invention.

FIG. 7 is an illustration of a patient having a nasogastric and endotracheal tube being held firm with an embodiment of the invention.

### DETAILED DESCRIPTION

Specific embodiments of the invention will now be described in detail with reference to the accompanying figures.

In the following detailed description of embodiments of the invention, specific details are set forth in order to provide a more thorough understanding of the invention. However, it will be apparent to one of ordinary skill in the art that the invention may be practiced without these specific details in lieu of substitutes. In other instances, well-known features have not been described in detail to avoid unnecessarily duplication and complication.

### OBJECTS AND ADVANTAGES

The present invention provides a simple device which allows practitioners and health care providers to attached and de-attached patient external tubes of various lengths and sizes. The device is simple and low cost, but more importantly, quicker to use and less wasteful on health care resources and care provider time.

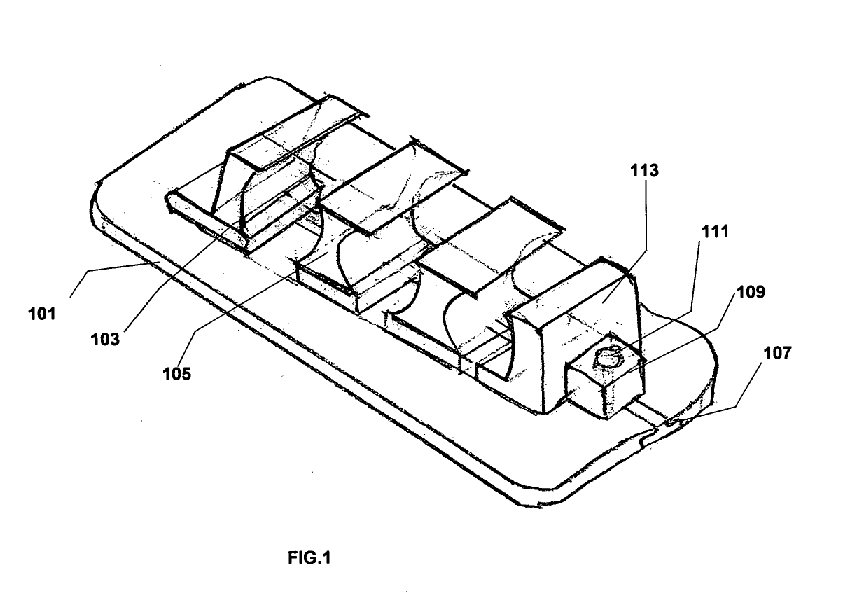

FIG. 1 is a isometric view of a tube coupler in accordance with an embodiment of the present invention. The embodiment rests on a base 101 in which a slider slot 107 allows for flexible tubing to be held firmly by friction from clamping elements 102 105 113, which are slipped into place to surround a flexible tube. The concave contours of the clamping elements 103 105 113 allow for some adjustment. The clamping elements are secured by a slidable anchoring block 109 which presses against the nearest slidable clamping element 113, exerting a frictional force constraining tube movement. The anchoring block 109 has a pin 111 mechanism for releasing and securing the anchor block 109. The base 101 is not limited in size and can be larger or smaller determined on the number of flexible tubes, size of tubes and outside anchor device and location.

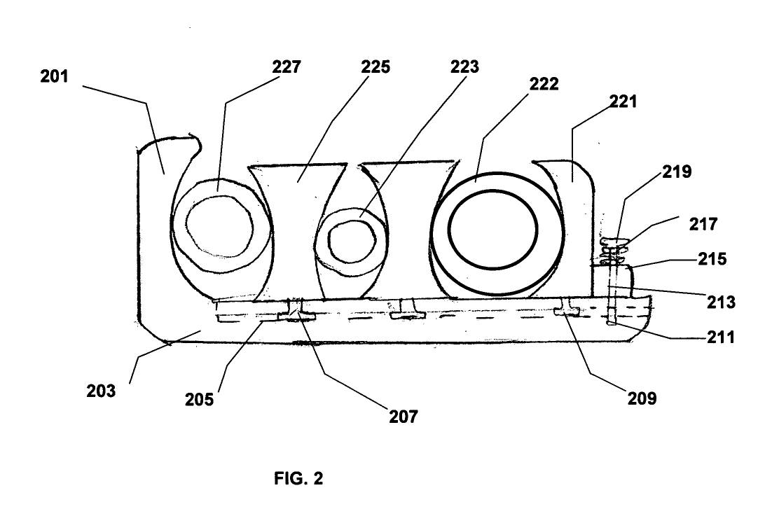

FIG. 2 is a side view drawing of a flexible tube coupler in accordance with an embodiment of the present invention. The base element 203 may curl into a part of a clamping element 201 and contains a slider channel 205 in which movable clamping elements 225 221 are positioned sliding clamping elements with rigid sliders 207 209 in the channel 205 to adjust clamping elements 225 221 positions to present a friction hold on various outside diameter size flexible tubes 222 223 227 pressed fit in a friction clamp to hold the tube 222 223 227 relative positions unchanging. At least one base end will have a block element 215 pressed or rigidly connected with a terminal clamping element 221. The block element 215 can have a pin 219 and pin-spring element 219 which allows the block element to slidably positioned in the channel 205 and anchored by pin stem 213 to a base hole 209 or slot. Many other anchoring mechanisms are possible

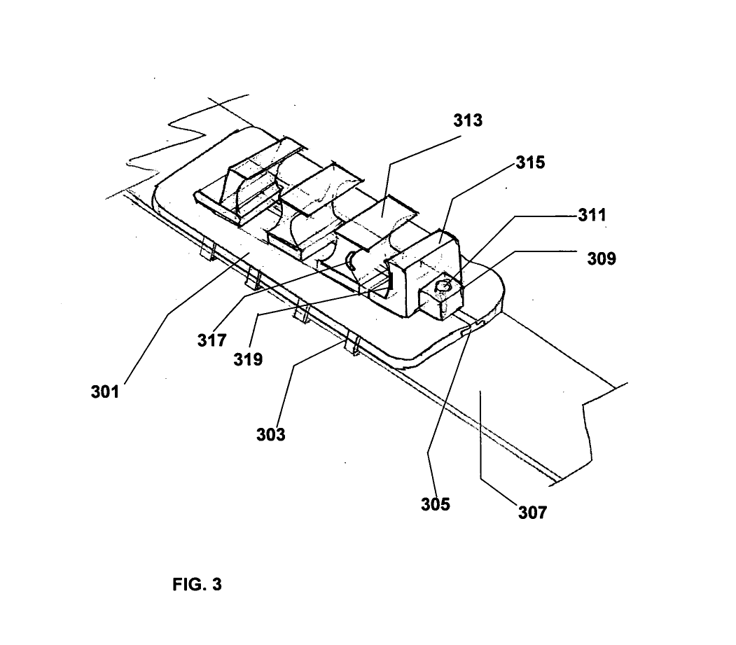

FIG. 3 is an isometric drawing of a flexible tube coupler on a belt in accordance with an embodiment of the present invention. The clamping elements 315 313, block 309 and spring pin slider 311 elements, slide channel 305 in base element 301 work as described in FIG. 2. The base element 301 is attached 303 to or is otherwise coupled to a belt 307 for constraining the coupler device to an additional anchor point, which may be a patients torso, body, appendage, pillow, etc. A patient may have a belt or wrap around the body, or appendage or a fixture, where the belt, 307 wrap or rigid element can secure the base 301 holding the flexible tubing immobile relative to the various tubes. Thus separate tubes can be added, inserted, removed, re-inserted and re-affixed by loosening the anchor 311 and sliding the block element 309 to make the necessary adjustment, closing the clamps 313 315 back around to friction hold the remaining tubes. In some embodiments small ink pads 317 319 for marking tube positions are placed at the edges of the clamps 313 315. The ink pads can be of different colors and/or color coded for specific tubes, functions, lengths, or positions. Flexible tubing with ink marking can be easily wiped clean for reuse, but certain applications will require water and wear resistant inks for more permanent markings. Rotating through clamps with different color ink pads can put a more permanent time history of marks through progressive placement process requiring flexible tubing applications in this fashion.

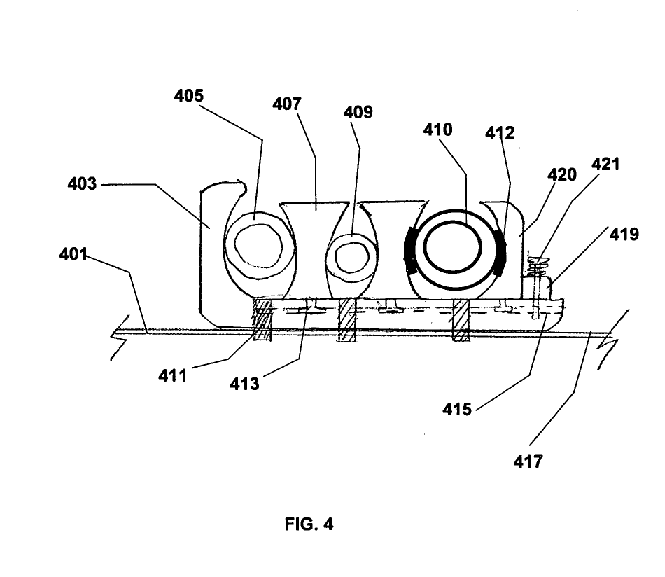

FIG. 4 is a side view drawing of a flexible tube coupler secured 411 to a belt 401 417 in accordance with an embodiment of the present invention. An inkpad strip 412 can be glued to a clamp element 420 edge, such that contact with a flexible tube 410 will ink a line onto the tube 410, demarking the position originally affixed. Repositioning or re-affixation can then be accomplished more precisely with the tube so marked, as the clamp edge can then be aligned with the tube marking, to achieve the precise original position after a tube removal or change. Different color inks or marking agents can be used, and color codes developed to distinguish tube, functions, changes, etc. The coupler base can integrally form one clamp edge 403 and contain a slide channel 415 for slidable clamping elements 407 420 along rigid slider attachment 413. A push pin 421 in slidable block 419 mechanism can be used to friction tighten the clamping elements 403 407 420 around flexible tubes 405 409 410 holding tube positions secure relative to each other. The belt 401 417 or tie down is then used to secure the coupler to a patient, patients apparel, or other convenient anchor.

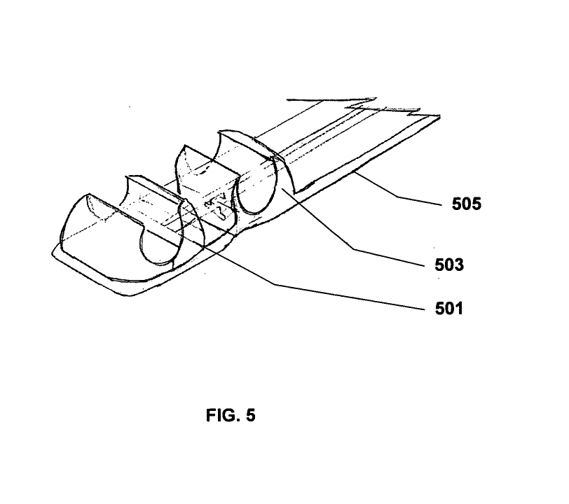

FIG. 5 is an isometric illustration of a one-piece clamping element coupler in accordance with an embodiment of the present invention. The outside diameters of flexible tubing is well known. Non adjustable flexible tube clamping elements 501 503 can be rigidly coupled or formed to a base 505 element which can then be attached to the patient's position an any conventional method. The cost of manufacturing fixed diameter tube couplers would be much less and provide a more simple solution available in some instances of patient care.

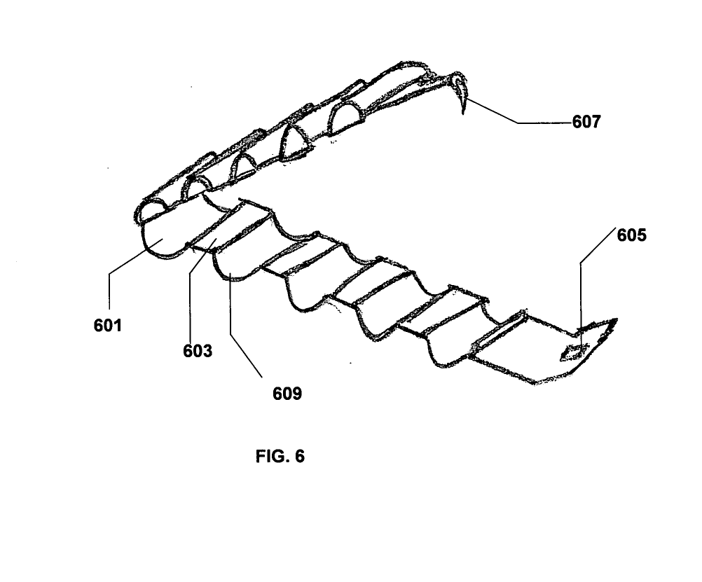

FIG. 6 is an isometric illustration of a rigid tube holder clasp in accordance with an embodiment of the present invention. A clasp 601 formed with tubular indentations 601 609 separated by clasp extensions 603 and secured by flexible insertion lock 605 607 mechanism can also firmly secure flexible tubes. The insertion locking mechanism 605 607 can be quickly opened and closed for tube placement and re-affixation.

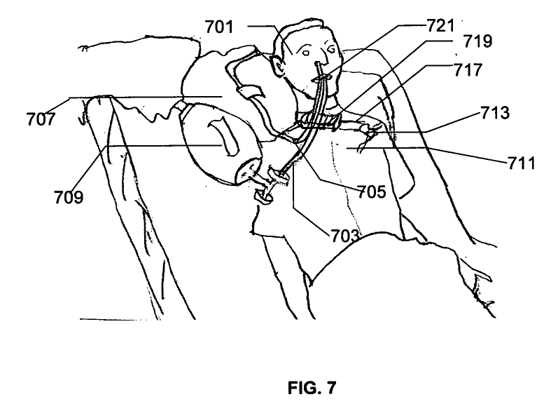

FIG. 7 is an illustration of a patient having inserted nasogastric and endotracheal tubes held secure with an embodiment of the invention. A patient 701 is shown lying in incline fashion with typical nasogastric 705 and endotracheal 703 tubes inserted in the patients face 721 orifices. The nasogastric reservoir 707 and the endotracheal reservoir 709 are shown attached and must be placed in stable positions to reduce tube 703 705 movement. The tubes 703 705 are held secure by an embodiment of the invention tube coupler 719 supported by a strap/belt 717 which is attached by clasp 713 to the patients outer garment 711. Only one clasp is visible as the other is anchored somewhat obscured by the patient's body. Thus the usual practice of taping is not shown or necessary.

Therefore, while the invention has been described with respect to a limited number of embodiments, those skilled in the art, having benefit of this invention, will appreciate that other embodiments can be devised which do not depart from the scope of the invention as disclosed herein. Accordingly, the scope of the invention should be limited only by the attached claims. Other aspects of the invention will be apparent from the following description and the appended claims.

What is claimed is:

1. A flexible tube coupler comprising:
a base element;
clamping elements coupled to base, clamping elements formed with curvature to partially surround a tubular object in a frictional fit; and
a clamp element base position securing mechanism;
whereby at least one flexible tube can be wedged between the clamping elements and rigidly coupled to the base element.

2. A tube coupler as in claim 1 further comprising slidably adjustable tube contour clamping elements on a base element slider.

3. A tube coupler as in claim 1 further comprising rigid clamping elements formed to friction fit known flexible tube outside diameters.

4. A tube coupler as in claim 1 further comprising a clasping base element with curved indentations on opposite arms of a clasp for securing tubes under a non tube materially deforming frictional mechanical pressure.

5. A tube coupler as in claim 1 further comprising the base and clamping element material made from a group of materials consisting of metal, plastic, composite, rubber and combinations.

6. A tube coupler as in claim 1 wherein the tubes coupled are of flexible plastic, rubber, nylon, synthetic or combinations.

7. A tube coupler as in claim 1 further comprising ink pads on clamping elements.

8. A tube coupler as in claim 1 further comprising a belt or other coupling mechanism securing the coupler base relative to the patient position.
